(19) United States
(12) Patent Application Publication
(10) Pub. No.: US 2015/0005262 A1
(43) Pub. Date: Jan. 1, 2015

(54) HYPOXIA ACTIVATED PRODRUGS AND MTOR INHIBITORS FOR TREATING CANCER

(71) Applicant: THRESHOLD PHARMACEUTICALS, INC., South San Francisco, CA (US)

(72) Inventors: Charles Hart, South San Francisco, CA (US); Jessica Sun, South San Francisco, CA (US); Fanying Meng, South San Francisco, CA (US)

(21) Appl. No.: 14/367,152

(22) PCT Filed: Dec. 20, 2012
(86) PCT No.: PCT/US2012/071070

§ 371 Date: Jun. 19, 2014

Related U.S. Application Data
(60) Provisional application No. 61/579,607, filed on Dec. 22, 2011.
(60) Provisional application No. 61/617,579, filed on Mar. 29, 2012.

(51) Int. Cl. A61K 31/675; A61K 31/436

(52) U.S. Cl. CPC A61K 31/675; A61K 31/436
USPC 514/94

(57) ABSTRACT

Cancer is treated by administration of a hypoxia activated prodrug in combination with an mTOR inhibitor.

### CROSS REFERENCE TO RELATED APPLICATIONS

This application claims priority under 35 U.S.C. 119(e) of U.S. application nos. 61/579,607, filed Dec. 22, 2011, and 61/617,579, filed Mar. 29, 2012, each of which is incorporated herein in its entirety by reference.

### FIELD OF THE INVENTION

The present invention provides methods for treating cancer, and pharmaceutical formulations and unit dose forms useful in those methods. The invention therefore relates to the fields of medicine and pharmacology.

### BACKGROUND OF THE INVENTION

TH-302 is a hypoxia activated prodrug in clinical development for the treatment of cancer. See PCT Publication Nos. 2007/002931; 2008/083101; 2010/048330; 2012/006032; and 2012/009288; PCT Patent Application Nos. PCT/US2012/031677, filed Mar. 30, 2012, and PCT/US2012/033671, filed Apr. 13, 2012; and U.S. Patent Application No. 61/593,249, filed on 31 January, 2012, each of which is incorporated herein by reference. TH-302 releases the DNA cross-linking bromo-isophosphoramidate (sometimes referred to as bromo-isophosphoramide) mustard (Br-IPM) under hypoxic conditions. TH-302 induces G₂/M arrest at low concentrations and a pan-cell cycle arrest at high concentrations.

The intracellular kinase mTOR plays a key role in multiple pathways that are important in cancer progression. Everolimus (marketed under the trade names Afinitor and Zortress) and temsirolimus (marketed under the trade name Torisel) directly target mTOR and reduce tumor cell proliferation, decrease tumor angiogenesis, and inhibit tumor cell metabolism.

However, mTOR inhibitors are problematic, because they can stimulate pathways that promote cancer cell growth and proliferation. For example, while impeding tumor growth by inhibiting the mTOR protein complex 1 (mTORC1), an mTOR inhibitor such as everolimus can activate the MAPK (mitogen-activated protein kinase) pathway, which may promote cancer cell survival. See Carracedo et al., J. Clin. Invest., 2008, 118(9): 3065-3074. MAPK signaling activates hypoxia inducible factor (HIF), which helps cancer cells survive in hypoxia. See, Sang et al., J. Biol. Chem., 2003, 278, 14013-14019.

Both everolimus and temsirolimus have been approved for the treatment of renal cell carcinoma (RCC), but such treatment rarely results in a cure. New and more effective therapies for RCC and other cancers are urgently needed.

The present invention meets this need.

### SUMMARY OF THE INVENTION

In a first aspect, the present invention provides a method of treating cancer, said method comprising administering to a patient in need of such treatment a therapeutically effective amount of a hypoxia activated prodrug in combination with a therapeutically effective amount of an mTOR inhibitor.

In various embodiments, the hypoxia activated prodrug is a compound of Formula I:

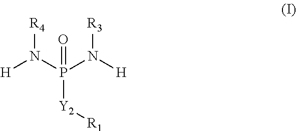

wherein Y₂ is O, S, NR₆, NCOR₆, or NSO₂R₆ wherein R₆ is C₁-C₆ alkyl, C₁-C₆ heteroalkyl, aryl, or heteroaryl; R₃ and R₄ are independently selected from the group consisting of 2-haloalkyl, 2-alkylsulfonyloxyalkyl, 2-heteroalkylsulfonyloxyalkyl, 2-arylsulfonyloxyalkyl, and 2-heteroalkylsulfonyloxyalkyl; R₁ has the formula L-Z₃; L is C(Z₁)₂; each Z₁ independently is hydrogen, halogen, C₁-C₆ alkyl, C₁-C₆ heteroalkyl, aryl, heteroaryl, C₃-C₈ cycloalkyl, heterocyclyl, C₁-C₆ acyl, C₁-C₆ heteroacyl, aroyl, or heteroaroyl; or L is:

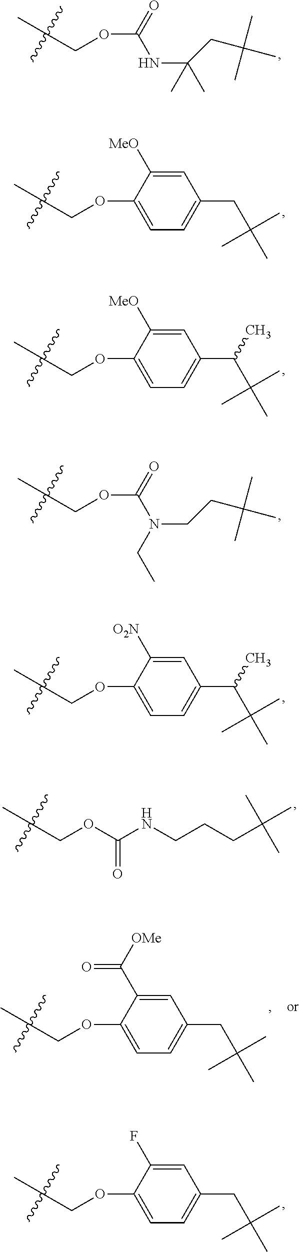

Z₃ is a bioreductive group having a formula selected from the group consisting of:

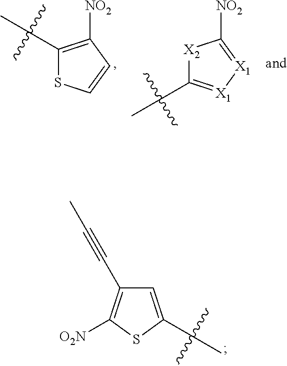

wherein each X₁ is independently N or CR₈; X₂ is NR₇, S, or O; each R₇ is independently C₁-C₆ alkyl, C₁-C₆ heteroalkyl, C₃-C₈ cycloalkyl, heterocyclyl, aryl or heteroaryl; and R₈ is independently hydrogen, halogen, cyano, CHF₂, CF₃, CO₂H, amino, C₁-C₆ alkyl, C₁-C₆ heteroalkyl, C₁-C₆ cycloalkyl, C₁-C₆ alkoxy, C₁-C₆ alkylamino, C₁-C₆ dialkylamino, aryl, CON(R₇)₂, C₁-C₆ acyl, C₁-C₆ heteroacyl, aroyl or heteroaroyl; or a pharmaceutically acceptable salt thereof. In various embodiments of the invention, the compound utilized in this invention is a compound of Formula I that is TH-281, TH-302, or TH-308 (structures provided below).

In one embodiment, the hypoxia activated prodrug is TH-302. In various preferred embodiments, the cancer treated is selected from the group consisting of renal cell carcinoma (RCC), neuroblastoma, and subependymal giant cell astrocytoma (SEGA).

In various embodiments, the TH-302 is administered at a dose and frequency described in PCT Publication Nos. 2007/002931; 2008/083101; 2010/048330; 2012/006032; and 2012/009288; and PCT Patent Application Nos. PCT/US2012/031677, filed Mar. 30, 2012, and PCT/US2012/033671, filed Apr. 13, 20112, each of which is incorporated herein by reference. In some embodiments, patients are selected for treatment and/or the efficacy of treatment is assessed in accordance with the methods described in U.S. Patent Application No. 61/593,249 filed on 31 Jan. 2012, incorporated herein by reference.

In various embodiments, the mTOR inhibitor is selected from the group consisting of AZD8055, BEZ235, deforolimus, everolimus, OSI-027, sirolimus, temsirolimus, and XL765. In various embodiments, the mTOR inhibitor is selected from the group consisting of everolimus and temsirolimus. In various embodiments, the mTOR inhibitor is administered at a daily amount and frequency approved for the treatment of various cancers by the FDA or as employed in clinical settings.

In one embodiment, the present invention provides an in vivo method of inhibiting growth of a tumor, comprising coadministering an effective amount of a compound of Formula 1 and an mTOR inhibitor to the tumor (i.e., to a cancer patient). In one embodiment, the tumor growth is inhibited completely, i.e., 100% TGI is observed. In another embodiment, there is tumor regression, i.e., TGI of >100% is observed. In other embodiments, the tumor growth is slowed. In one embodiment, the mTOR inhibitor is everolimus or temsirolimus. In one embodiment, the compound of Formula 1 is TH-302. In one embodiment, TH-302 is administered once daily, for five consecutive days a week. In other embodiments, TH-302 is administered no more than once a week. In any of these embodiments, TH-302 therapy can be continued for multiple weeks or months.

In one embodiment, the present invention provides an in vivo method of reducing tumor hypoxia in a tumor treated with an mTOR inhibitor, the method comprising coadministering an effective amount of a compound of Formula 1 to the tumor (i.e., to a cancer patient). In one embodiment, the tumor hypoxia is reduced by up to 5%, up to 10%, or by up to 15% or more, i.e., 25%, 50% or 95%. As used herein, “a tumor treated with an mTOR inhibitor” refers to a tumor, one or more tumor cells of which were contacted with, are in contact with, or are to be contacted with an mTOR inhibitor. In one embodiment, the mTOR inhibitor is everolimus or temsirolimus. In one embodiment, the compound of Formula 1 is TH-302. In one embodiment, TH-302 is administered once daily, for five consecutive days a week. In other embodiments, TH-302 is administered no more than once a week. In any of these embodiments, TH-302 therapy can be continued for multiple weeks or months.

Coadministering, as used herein, contemplates that the two drugs coadministered exert their pharmacological effect in a tumor cell at the same time; such coadministration can be achieved by simultaneous, contemporaneous, or sequential administration of the two drugs.

In a second aspect, the present invention provides pharmaceutical formulations and unit dose forms suitable for use in the methods of the present invention. In one embodiment, the hypoxia activated prodrug and the mTOR inhibitor are formulated separately in distinct unit dose forms. In another embodiment, the hypoxia activated prodrug and the mTOR inhibitor are formulated together in an admixture or other combination pharmaceutical formulation and combination unit dose forms. In various embodiments, the hypoxia activated prodrug is TH-302.

### BRIEF DESCRIPTION OF THE FIGURES

FIG. 1 shows the structures of illustrative mTOR inhibitors useful in the practice of this invention.

### DETAILED DESCRIPTION OF THE INVENTION

The practice of the present invention may include the use of conventional techniques of biochemistry, cell biology, immunology, organic chemistry, medicine, microbiology, molecular biology (including recombinant techniques), and pharmacology, which are within the skill of the art.

### DEFINITIONS

In this specification and in the claims that follow, reference will be made to a number of terms that have the meanings below. All numerical designations, e.g., pH, temperature, time, concentration, and weight, including ranges of each thereof, are approximations that typically may be varied (+) or (−) by increments of 0.1, 1.0, or 10.0, as appropriate. All numerical designations may be understood as preceded by the term “about”. Reagents described herein are exemplary and equivalents of such may be known in the art.

The singular form “a”, “an”, and “the” includes plural references unless the context clearly dictates otherwise.

The term “comprising” means any recited elements are necessarily included and other elements may optionally be included. “Consisting essentially of” means any recited elements are necessarily included, elements that would materially affect the basic and novel characteristics of the listed elements are excluded, and other elements may optionally be included. “Consisting of” means that all elements other than those listed are excluded. Embodiments defined by each of these terms are within the scope of this invention.

Certain terms related to Formula I are defined below.

“Acyl” refers to —CO-alkyl, wherein alkyl is as defined here.

“Aroyl” refers to —CO-aryl, wherein aryl is as defined here.

“Alkoxy” refers to —O-alkyl, wherein alkyl is as defined here.

“Alkenyl” refers to a linear monovalent hydrocarbon radical or a branched monovalent hydrocarbon radical having the number of carbon atoms indicated in the prefix and containing at least one double bond, but no more than three double bonds. For example, (C₂-C₆)alkenyl includes, ethenyl, propenyl, 1,3-butadienyl and the like. Alkenyl can be optionally substituted with substituents, including for example, deuterium (“D”), hydroxyl, amino, mono or di(C₁-C₆)alkyl amino, halo, C₂-C₆ alkenyl ether, cyano, nitro, ethynyl, C₁-C₆ alkoxy, C₁-C₆ alkylthio, —COOH, —CONH₂, mono- or di(C₁-C₆)alkylcarboxamido, —SO₂NH₂, —OSO₂—(C₁-C₆)alkyl, mono or di(C₁-C₆) alkylsulfonamido, aryl, heteroaryl, alkyl or heteroalkylsulfonyloxy, and aryl or heteroarylsulfonyloxy.

“Alkyl” refers to a linear saturated monovalent hydrocarbon radical or a branched saturated monovalent hydrocarbon radical having the number of carbon atoms indicated in the prefix. (C₁-C₆)alkyl can be optionally substituted with substituents, including for example, deuterium (“D”), hydroxyl, amino, mono or di(C₁-C₆) alkyl amino, halo, C₂-C₆ alkenyl ether, cyano, nitro, ethenyl, ethynyl, C₁-C₆ alkoxy, C₁-C₆ alkylthio, —COOH, —CONH₂, mono- or di(C₁-C₆)alkylcarboxamido, —SO₂NH₂, —OSO₂—(C₁-C₆)alkyl, mono or di(C₁-C₆) alkylsulfonamido, aryl, heteroaryl, alkylsulfonyloxy, heteroalkylsulfonyloxy, arylsulfonyloxy or heteroarylsulfonyloxy.

The prefixes (C₁-C_{qq}), C_{1-qq}, and C₁-C_{qq}, wherein qq is an integer from 2-20, have the same meaning. For example, (C₁-C₆)alkyl, C₁₋₆ alkyl, or C₁-C₆ alkyl includes methyl, ethyl, n-propyl, 2-propyl, n-butyl, 2-butyl, tert-butyl, pentyl, and the like. For each of the definitions herein (e.g., alkyl, alkenyl, alkoxy, etc.), when a prefix is not included to indicate the number of main chain carbon atoms in an alkyl portion, the radical or portion thereof will have six or fewer main chain carbon atoms.

“Alkylamino” or mono-alkylamino refers to —NH-alkyl, wherein alkyl is as defined here.

“Alkynyl” refers to a linear monovalent hydrocarbon radical or a branched monovalent hydrocarbon radical having the number of carbon atoms indicated in the prefix and containing at least one triple bond, but no more than two triple bonds. For example, (C₂-C₆)alkynyl includes, ethynyl, propynyl, and the like. Alkynyl can be optionally substituted with substituents, including for example, deuterium (“D”), hydroxyl, amino, mono or di(C₁-C₆)alkyl amino, halo, C₂-C₆ alkenyl ether, cyano, nitro, ethenyl, C₁-C₆ alkoxy, C₁-C₆ alkylthio, —COOH, —CONH₂, mono- or di(C₁-C₆)alkylcarboxamido, —SO₂NH₂, —OSO₂—(C₁-C₆)alkyl, mono or di(C₁-C₆)alkylsulfonamido, aryl, heteroaryl, alkyl or heteroalkylsulfonyloxy, and aryl or heteroarylsulfonyloxy.

“Aryl” refers to a monovalent monocyclic or bicyclic aromatic hydrocarbon radical of 6 to 10 ring atoms which is substituted independently with one to eight substituents, e.g., one, two, three, four of five substituents selected from deuterium (“D”), alkyl, cycloalkyl, cycloalkylalkyl, halo, nitro, cyano, hydroxyl, alkoxy, amino, acylamino, mono-alkylamino, di-alkylamino, haloalkyl, haloalkoxy, heteroalkyl, COR (where R is hydrogen, alkyl, cycloalkyl, cycloalkyl-alkyl, phenyl or phenylalkyl), —(CR′R″)ₙ—COOR (where n is an integer from 0 to 5, R′ and R″ are independently hydrogen or alkyl, and R is hydrogen, alkyl, cycloalkyl, cycloalkylalkyl, phenyl or phenylalkyl) or —(CR′R″)ₙ—CONR^{x}R^{y} (where n is an integer from 0 to 5, R′ and R″ are independently hydrogen or alkyl, and R^{x} and R^{y} are independently selected from hydrogen, alkyl, cycloalkyl, cycloalkylalkyl, phenyl or phenylalkyl). In one embodiment, R^{x} and R^{y} together is cycloalkyl or heterocyclyl. More specifically, the term aryl includes, but is not limited to, phenyl, biphenyl, 1-naphthyl, and 2-naphthyl, and the substituted forms thereof.

“Cycloalkyl” refers to a monovalent cyclic hydrocarbon radical of three to seven ring carbons. The cycloalkyl group can have one or more double bonds and can also be optionally substituted independently with one, two, three or four substituents selected from alkyl, optionally substituted phenyl, or —C(O)R^{z} (where R^{z} is hydrogen, alkyl, haloalkyl, amino, mono-alkylamino, di-alkylamino, hydroxyl, alkoxy, or optionally substituted phenyl). More specifically, the term cycloalkyl includes, for example, cyclopropyl, cyclohexyl, cyclohexenyl, phenylcyclohexyl, 4-carboxycyclohexyl, 2-carboxamidocyclohexenyl, 2-dimethylaminocarbonyl-cyclohexyl, and the like.

“Dialkylamino” or di-alkylamino refers to —N(alkyl)₂, wherein alkyl is as defined here.

“Heteroalkyl” refers to an alkyl radical as defined here with one, two or three substituents independently selected from cyano, —OR^{w}, —NR^{x}R^{y}, and —S(O)ₚR^{z} (where p is an integer from 0 to 2), with the understanding that the point of attachment of the heteroalkyl radical is through a carbon atom of the heteroalkyl radical. R^{w} is hydrogen, alkyl, cycloalkyl, cycloalkyl-alkyl, aryl, aralkyl, alkoxycarbonyl, aryloxycarbonyl, carboxamido, or mono- or di-alkylcarbamoyl. R^{x} is hydrogen, alkyl, cycloalkyl, cycloalkyl-alkyl, aryl or araalkyl. R^{y} is hydrogen, alkyl, cycloalkyl, cycloalkyl-alkyl, aryl, araalkyl, alkoxycarbonyl, aryloxycarbonyl, carboxamido, mono- or di-alkylcarbamoyl or alkylsulfonyl. R^{z} is hydrogen (provided that p is 0), alkyl, cycloalkyl, cycloalkyl-alkyl, aryl, araalkyl, amino, mono-alkylamino, di-alkylamino, or hydroxyalkyl. Representative examples include, for example, 2-hydroxyethyl, 2,3-dihydroxypropyl, 2-methoxyethyl, benzyloxymethyl, 2-cyanoethyl, and 2-methylsulfonyl-ethyl. For each of the above, R^{w}, R^{x}, R^{y}, and R^{z} can be further substituted by amino, halo, fluoro, alkylamino, di-alkylamino, OH or alkoxy. Additionally, the prefix indicating the number of carbon atoms (e.g., C₁-C₁₀) refers to the total number of carbon atoms in the portion of the heteroalkyl group exclusive of the cyano, —OR^{w}, —NR^{x}R^{y}, or —S(O)ₚR^{z} portions. In one embodiment, R^{x} and R^{y} together is cycloalkyl or heterocyclyl.

“Heteroaryl” refers to a monovalent monocyclic, bicyclic or tricyclic radical of 5 to 12 ring atoms having at least one aromatic ring containing one, two, or three ring heteroatoms selected from N, O, or S, the remaining ring atoms being C, with the understanding that the attachment point of the heteroaryl radical will be on an aromatic ring. The heteroaryl ring is optionally substituted independently with one to eight substituents, preferably one, two, three or four substituents, selected from alkyl, cycloalkyl, cycloalkyl-alkyl, halo, nitro, cyano, hydroxyl, alkoxy, amino, acylamino, mono-alkylamino, di-alkylamino, haloalkyl, haloalkoxy, heteroalkyl, —COR (where R is hydrogen, alkyl, phenyl or phenylalkyl, —(CR′R″)ₙ—COOR (where n is an integer from 0 to 5, R′ and R″ are independently hydrogen or alkyl, and R is hydrogen, alkyl, cycloalkyl, cycloalkyl-alkyl, phenyl or phenylalkyl), or —(CR′R″)ₙ—CONR^{x}R^{y} (where n is an integer from 0 to 5, R′ and R″ are independently hydrogen or alkyl, and R^{x} and R^{y} are, independently of each other, hydrogen, alkyl, cycloalkyl, cycloalkyl-alkyl, phenyl or phenylalkyl). In one embodiment, R^{x} and R^{y} together is cycloalkyl or heterocyclyl. More specifically the term heteroaryl includes, but is not limited to, pyridyl, furanyl, thienyl, thiazolyl, isothiazolyl, triazolyl, imidazolyl, isoxazolyl, pyrrolyl, pyrazolyl, pyridazinyl, pyrimidinyl, benzofuranyl, tetrahydrobenzofuranyl, isobenzofuranyl, benzothiazolyl, benzoisothiazolyl, benzotriazolyl, indolyl, isoindolyl, benzoxazolyl, quinolyl, tetrahydroquinolinyl, isoquinolyl, benzimidazolyl, benzisoxazolyl, benzothienyl, indazolyl, pyrrolopyrymidinyl, indolizinyl, pyrazolopyridinyl, triazolopyridinyl, pyrazolopyrimidinyl, triazolopyrimidinyl, pyrrolotriazinyl, pyrazolotriazinyl, triazolotriazinyl, pyrazolotetrazinyl, hexaaza-indenly, and heptaaza-indenyl and the derivatives thereof. Unless indicated otherwise, the arrangement of the hetero atoms within the ring can be any arrangement allowed by the bonding characteristics of the constituent ring atoms.

“Heterocyclyl” or “cycloheteroalkyl” refers to a saturated or unsaturated non-aromatic cyclic radical of 3 to 8 ring atoms in which one to four ring atoms are heteroatoms selected from O, NR (where R is hydrogen, alkyl, cycloalkyl, cycloalkylalkyl, phenyl or phenylalkyl), P(═O)OR^{w}, or S(O)ₚ (where p is an integer from 0 to 2), the remaining ring atoms being C, wherein one or two C atoms can optionally be replaced by a carbonyl group. The heterocyclyl ring can be optionally substituted independently with one, two, three or four substituents selected from alkyl, aryl, arylalkyl, heteroaryl, heteroarylalkyl, cycloalkyl, cycloalkylalkyl, halo, nitro, cyano, hydroxyl, alkoxy, amino, mono-alkylamino, di-alkylamino, haloalkyl, haloalkoxy, —COR (where R is hydrogen, alkyl, cycloalkyl, cycloalkylalkyl, phenyl or phenylalkyl), —(CR′R″)ₙ—COOR (n is an integer from 0 to 5, R′ and R″ are independently hydrogen or alkyl, and R is hydrogen, alkyl, cycloalkyl, cycloalkylalkyl, phenyl or phenylalkyl), or —(CR′R″)ₙ—CONR^{x}R^{y} (where n is an integer from 0 to 5, R′ and R″ are independently hydrogen or alkyl, R^{x} and R^{y} are, independently of each other, hydrogen, alkyl, cycloalkyl, cycloalkylalkyl, phenyl or phenylalkyl). More specifically, the term heterocyclyl includes, but is not limited to, tetrahydropyranyl, N-methylpiperidin-3-yl, N-methylpyrrolidin-3-yl, 2-pyrrolidon-1-yl, pyrrolidinyl, piperidinyl, morpholinyl, tetrahydrofuranyl, tetrahydrothiofuranyl, 1,1-dioxo-hexahydro-1Δ⁶-thiopyran-4-yl, tetrahydroimidazo[4,5-c]pyridinyl, imidazolinyl, piperazinyl, and piperidin-2-yl and the derivatives thereof. The prefix indicating the number of carbon atoms (e.g., C₃-C₁₀) refers to the total number of carbon atoms in the portion of the cycloheteroalkyl or heterocyclyl group exclusive of the number of heteroatoms.

“Heteroacyl” refers to —CO-heteroalkyl, wherein heteroalkyl is as defined here.

“Heteroaroyl” refers to —CO-heteroayl, wherein heteroaryl is as defined here.

“Rₛᵤₗ sulfonyloxy” refers to Rₛᵤₗ—S(═O)₂—O— and includes alkylsulfonyloxy, heteroakylsulfonyloxy, cycloalkylsulfonyloxy, heterocyclylsulfonyloxy, arylsulfonyloxy and heteroarylsulfonyloxy wherein Rₛᵤₗ is alkyl, heteroakyl, cycloalkyl, heterocyclyl, aryl and heteroaryl respectively, and wherein alkyl, heteroakyl, cycloalkyl, heterocyclyl, aryl and heteroaryl are as defined here. Examples of alkylsulfonyloxy include Me-S(═O)₂—O—, Et-S(═O)₂—O—, CF₃—S(═O)₂—O— and the like, and examples of arylsulfonyloxy include:

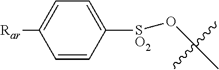

wherein Rₐᵣ is H, methyl, or bromo.

“Substituents” refer to, along with substituents particularly described in the definition of each of the groups above, those selected from: deuterieum, -halogen, —OR′, —NR′R″, —SR′, —SiR′R″R′″, —OC(O)R′, —C(O)R′, —CO₂R′, —CONR′R″, —OC(O)NR′R″, —NR″C(O)R′, —NR′—C(O)NR″R′″, —NR″C(O)₂R′, —NH—C(NH₂)═NH, —NR′C(NH)═NH, —NH—C(NH₂)═NR′, —S(O)R′, —S(O)₂R′, —S(O)₂NR′R″, —NR'S(O)₂R″, —CN, —NO₂, —R′, —N₃, perfluoro(C₁-C₄)alkoxy, and perfluoro(C₁-C₄)alkyl, in a number ranging from zero to the total number of open valences on the radical; and where R′, R″ and R′″ are independently selected from hydrogen, C₁₋₈ alkyl, C₃₋₆ cycloalkyl, C₂₋₈ alkenyl, C₂₋₈ alkynyl, unsubstituted aryl and heteroaryl, (unsubstituted aryl)-C₁-₄ alkyl, and unsubstituted aryloxy-C₁-₄ alkyl, aryl substituted with 1-3 halogens, unsubstituted C₁-8 alkyl, C₁-₈alkoxy or C₁-₈ thioalkoxy groups, or unsubstituted aryl-C₁-₄ alkyl groups. When R′ and R″ are attached to the same nitrogen atom, they can be combined with the nitrogen atom to form a 3-, 4-, 5-, 6-, or 7-membered ring. For example, —NR′R″ is meant to include 1-pyrrolidinyl and 4-morpholinyl. Other suitable substituents include each of the above aryl substituents attached to a ring atom by an alkylene tether of from 1-4 carbon atoms. Two of the substituents on adjacent atoms of the aryl or heteroaryl ring may optionally be replaced with a substituent of the formula -T²-C(O)(CH₂)_{q}—U³—, wherein T² and U³ are independently —NH—, —O—, —CH₂— or a single bond, and q is an integer of from 0 to 2. Alternatively, two of the substituents on adjacent atoms of the aryl or heteroaryl ring may optionally be replaced with a substituent of the formula -A-(CH₂), —B—, wherein A and B are independently —CH₂—, —O—, —NH—, —S—, —S(O)—, —S(O)₂—, —S(O)₂NR′— or a single bond, and r is an integer of from 1 to 3. One of the single bonds of the new ring so formed may optionally be replaced with a double bond. Alternatively, two of the substituents on adjacent atoms of the aryl or heteroaryl ring may optionally be replaced with a substituent of the formula —(CH₂)ₛ—X⁵—(CH₂)ₜ—, wherein s and t are independently integers of from 0 to 3, and X⁵ is —O—, —NR′—, —S—, —S(O)—, —S(O)₂—, or —S(O)₂NR′—. The substituent R′ in —NR′— and —S(O)₂NR′— is selected from hydrogen or unsubstituted C₁-₆ alkyl.

Certain compounds utilized in the present invention possess asymmetric carbon atoms (optical centers) or double bonds; the racemates, diastereomers, geometric isomers, regioisomers and individual isomers (e.g., separate enantiomers) are all intended to be encompassed within the scope of the present invention. The compounds of the present invention may also contain unnatural proportions of atomic isotopes at one or more of the atoms that constitute such compounds. For example, the compounds may be radiolabeled with radioactive isotopes, such as for example, and without limitation, tritium (³H), iodine-125 (¹²⁵I), or carbon-14 (¹⁴C). All isotopic variations of the compounds of the present invention, whether radioactive or not, are intended to be encompassed within the scope of the present invention.

Other terms related to this invention are defined below.

“Administering” or “administration of” a drug to a patient (and grammatical equivalents of this phrase) refers to direct administration, which may be administration to a patient by a medical professional or may be self-administration, and/or indirect administration, which may be the act of prescribing a drug. For example, a physician who instructs a patient to self-administer a drug and/or provides a patient with a prescription for a drug is administering the drug to the patient.

“BD” or “bid” refers to twice daily dosing.

“Cancer” refers to malignant solid tumors of potentially unlimited growth, as well as various blood cancers that may originate from cancer stem cells in the bone marrow, which can expand locally by invasion and systemically by metastasis. Examples of cancers include, but are not limited to, cancer of the adrenal gland, bone, brain, breast, bronchi, colon and/or rectum, gallbladder, gastrointestinal tract, head and neck, kidneys, larynx, liver, lung, neural tissue, pancreas, prostate, parathyroid, skin, stomach, and thyroid. Other examples of cancers include, adenocarcinoma, adenoma, basal cell carcinoma, cervical dysplasia and in situ carcinoma, Ewing's sarcoma, epidermoid carcinomas, giant cell tumor, glioblastoma multiforma, hairy-cell tumor, intestinal ganglioneuroma, hyperplastic corneal nerve tumor, islet cell carcinoma, Kaposi's sarcoma, leiomyoma, leukemias, lymphomas, malignant carcinoid, malignant melanomas, malignant hypercalcemia, marfanoid habitus tumor, medullary carcinoma, metastatic skin carcinoma, mucosal neuroma, myelodisplastic syndrome, myeloma, mycosis fungoides, neuroblastoma, osteosarcoma, osteogenic and other sarcoma, ovarian tumor, pheochromocytoma, polycythermia vera, primary brain tumor, small-cell lung tumor, squamous cell carcinoma of both ulcerating and papillary type, seminoma, soft tissue sarcoma, retinoblastoma, rhabdomyosarcoma, renal cell tumor or renal cell carcinoma, veticulum cell sarcoma, and Wilm's tumor. Examples of cancers also include astrocytoma, a gastrointestinal stromal tumor (GIST), a glioma or glioblastoma, renal cell carcinoma (RCC), hepatocellular carcinoma (HCC), and a pancreatic neuroendocrine cancer.

“Combination therapy” or “combination treatment” refers to the use of two or more drugs in therapy, i.e., use of a hypoxia activated prodrug as described herein together with one or more mTOR inhibitors and optionally one or more other anti-cancer agents, used to treat cancer. Administration in “combination” refers to the administration of two agents (e.g., a hypoxia activated prodrug and an mTOR inhibitor for treating cancer) in any manner in which the pharmacological effects of both are manifest in the patient at the same time. Thus, administration in combination does not require that a single pharmaceutical composition, the same dosage form, or the same route of administration be used for administration of both agents or that the two agents be administered at precisely the same time. For example, and without limitation, it is contemplated that an mTOR inhibitor can be administered with a hypoxia activated prodrug in accordance with the present invention in combination therapy.

“Hyperproliferative disease” refers to a disease characterized by cellular hyperproliferation (e.g., an abnormally increased rate or amount of cellular proliferation). Cancer is a hyperproliferative disease. Examples of hyperproliferative diseases other than cancer include, but are not limited to, allergic angiitis and granulomatosis (Churg-Strauss disease), asbestosis, asthma, atrophic gastritis, benign prostatic hyperplasia, bullous pemphigoid, coeliac disease, chronic bronchitis and chronic obstructive airway disease, chronic sinusitis, Crohn's disease, demyelinating neuropathies, dermatomyositis, eczema including atopic dermatitis, eustachean tube diseases, giant cell arteritis, graft rejection, hypersensitivity pneumonitis, hypersensitivity vasculitis (Henoch-Schonlein purpura), irritant dermatitis, inflammatory hemolytic anemia, inflammatory neutropenia, inflammatory bowel disease, Kawasaki's disease, multiple sclerosis, myocarditis, myositis, nasal polyps, nasolacrimal duct diseases, neoplastic vasculitis, pancreatitis, pemphigus vulgaris, primary glomerulonephritis, psoriasis, periodontal disease, polycystic kidney disease, polyarteritis nodosa, polyangitis overlap syndrome, primary sclerosing cholangitis, rheumatoid arthritis, serum sickness, surgical adhesions, stenosis or restenosis, scleritis, scleroderma, strictures of bile ducts, strictures (of duodenum, small bowel, and colon), silicosis and other forms of pneumoconiosis, type I diabetes, ulcerative colitis, ulcerative proctitis, vasculitis associated with connective tissue disorders, vasculitis associated with congenital deficiencies of the complement system, vasculitis of the central nervous system, and Wegener's granulomatosis.

“Hypoxia activated prodrug” refers to a drug that is less active or inactive under normoxia than under hypoxia or anoxia. Hypoxia activated prodrugs include drugs that are activated by a variety of reducing agents, including glutathione, GSH, NADH, and NADPH, and reducing enzymes, including without limitation single electron transferring enzymes (such as cytochrome P450 reductases) and two electron transferring (or hydride transferring) enzymes (see U.S. Pat. App. Pub. Nos. 2005/0256191, 2007/0032455, and 2009/0136521, and PCT Pub. Nos. 2000/064864, 2004/087075, and 2007/002931, each of which is incorporated herein by reference). The hypoxia activated prodrugs useful in the methods of the present invention are compounds of Formula I, including but not limited to compounds where Z₃, as defined by that formula, is a 2-nitroimidazole moiety. Examples of particular hypoxia activated prodrugs useful in the methods of the invention include without limitation TH-281, TH-302, and TH-308. Methods of synthesizing, formulating, and using TH-302 and other compounds of Formula I are described in the various patent publications and applications referenced in the “Background of the Invention”, above, which are incorporated herein by reference.

The term “mTOR” or the mammalian target of rapamycin, also known as the mechanistic target of rapamycin, FK506 binding protein, or rapamycin associated protein 1 (FRAP1), refers to a protein which is a serine/threonine protein kinase that regulates cell growth, cell proliferation, cell motility, cell survival, protein synthesis, and transcription. In humans, mTOR is encoded by the FRAP1 gene.

The term “mTOR inhibitor” refers to an inhibitor of mTOR, and includes pharmaceutically acceptable salts thereof. Examples of mTOR inhibitors include without limitation rapamycin (sirolimus), shown structurally below, and rapamycin derivatives, for example those containing water soluble esters and ethers of the hydroxy group of rapamycin shown by an arrow below:

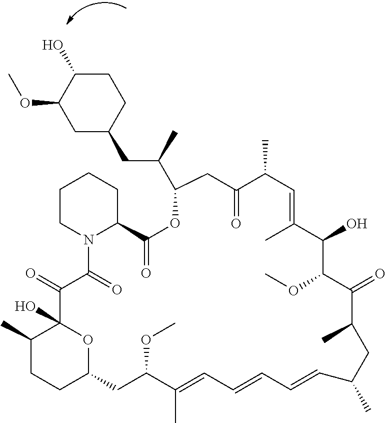 [SMILES: CO[C@H]1C[C@@H]2CC[C@@H](C)[C@@](O)(O2)C(=O)C(=O)N2CCCC[C@H]2C(=O)O[C@H]([C@H](C)C[C@@H]2CC[C@@H](O)[C@H](OC)C2)CC(=O)[C@H](C)/C=C(\C)[C@@H](O)[C@@H](OC)C(=O)[C@H](C)C[C@H](C)/C=C/C=C/C=C/1C]

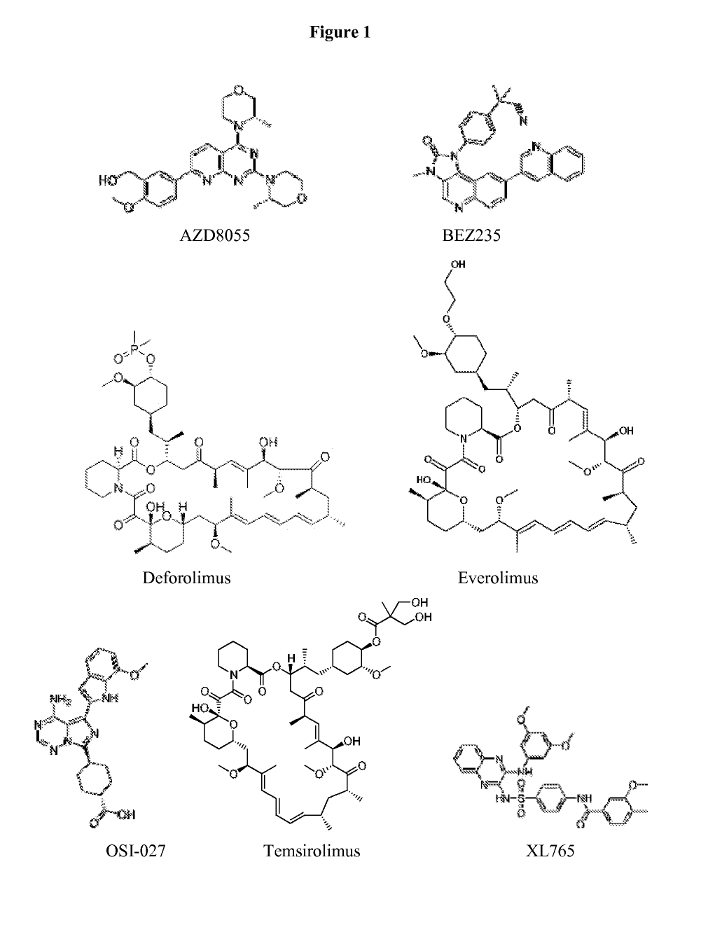

Such water soluble esters include, in place of the hydrogen atom of the hydroxy group, without limitation, a —CO—R_{E} or —PO(R_{E})₂ moiety, where R_{E} is C₁-C₁₀ alkyl optionally substituted with up to six hydroxy groups or C₃-C₈ cycloalkyl optionally substituted with up to six hydroxy groups. Such water soluble ethers include, in place of the hydrogen atom of the hydroxy group, without limitation, an R_{E} moiety, where R_{E} is defined as above. Other non-limiting examples of mTOR inhibitors include AZD8055, BEZ235 (NVP-BEZ235), chrysophanic acid (chrysophanol), deforolimus (ridaforolimus or AP23573), everolimus (RAD001), GSK1059615, GSK2126458, KU-0063794, NU7441, OSI-027, Palomid 529 (P529), PI-103, PKI-587, PP242, rapamycin, temsirolimus (Torisel), WAY-600, WYE-125132, WYE-354, WYE-687, and XL765. The structures of various illustrative mTOR inhibitors useful in the methods of the invention are provided in FIG. 1.

“Patient” or “subject” refers to mammals, particularly humans, and so includes animals of veterinary and research interest, such as simians, cattle, horses, dogs, cats, and rodents with cancer or another hyperproliferative disease.

“Pharmaceutically acceptable salt” refers to pharmaceutically acceptable salts derived from a variety of organic and inorganic counter ions well known in the art that include, by way of example only, sodium, potassium, calcium, magnesium, ammonium, and tetraalkylammonium, and when the molecule contains a basic functionality, salts of organic or inorganic acids, such as hydrochloride, hydrobromide, tartrate, mesylate, acetate, maleate, and oxalate. Suitable salts include those described in Stahl and Wermuth (Eds.), Handbook of Pharmaceutical Salts Properties, Selection, and Use; 2002.

QnD or qnd refers to drug administration once every n days. For example, QD (or qd) refers to once every day or once daily dosing, Q2D (or q2d) refers to a dosing once every two days, Q7D refers to a dosing once every 7 days or once a week, Q5D refers to dosing once every 5 days.

“Reduction” of a symptom or symptoms (and grammatical equivalents of this phrase) refers to decreasing the severity or frequency of the symptom(s), or elimination of the symptom(s).

“Relapsed or refractory” refers to a type of cancer that is resistant to treatment with an agent, such as mTOR inhibitor or a hypoxia activated prodrug, or responds to treatment with an agent but recurs with or without being resistant to that agent.

TH-281 refers to the compound of formula:

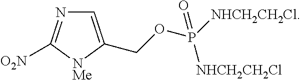

TH-302 refers to the compound of formula:

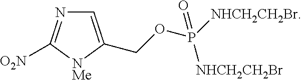

TH-308 refers to the compound of formula:

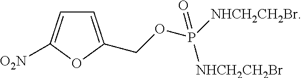

“Therapeutically effective amount” of a drug or an agent refers to an amount of the drug or the agent that, when administered to a patient with cancer or another hyperproliferative disease, will have the intended therapeutic effect, e.g., alleviation, amelioration, palliation or elimination of one or more manifestations of cancer or another hyperproliferative disease in the patient. A therapeutic effect does not necessarily occur by administration of one dose, and may occur only after administration of a series of doses. Thus, a therapeutically effective amount may be administered in one or more administrations.

“Treating” or “treatment of” a condition or patient refers to taking steps to obtain beneficial or desired results, including clinical results. For purposes of this invention, beneficial or desired clinical results include, but are not limited to, alleviation or amelioration of one or more symptoms of cancer or another hyperproliferative disease including conditional survival and reduction of tumor load or volume; diminishment of extent of disease; delay or slowing of disease progression; amelioration, palliation, or stabilization of the disease state; or other beneficial results.

The present invention arises in part from the discovery that the pharmacological inhibition of mTOR can potentiate the efficacy of TH-302 and other drugs of Formula I, when administered in accordance with the methods of the invention. As demonstrated in the examples below, administration of everolimus in combination with TH-302 and administration of temsirolimus in combination with TH-302 demonstrated superior efficacy over administration of any of the agents alone in animal models of cancer, including RCC and neuroblastoma models. Administration of a hypoxia activated prodrug such as TH-302 in accordance with the present invention in combination with everolimus or temsirolimus can therefore improve cancer treatment outcomes.

As demonstrated in animal models described in the examples below, administration of TH-302 in combination with everolimus improved the efficacy profile versus the use of either agent in monotherapy. As described in Example 1, below, in a Caki-1 model of RCC, 87% tumor growth inhibition (TGI) was achieved in the combination therapy group versus 52% TGI from everolimus monotherapy or 57% TGI from TH-302 monotherapy. In the 786-0 model of RCC described in Example 1, below, everolimus monotherapy provided 49% TGI, while TH-302 monotherapy did not demonstrate statistically significant TGI. However, when TH-302 was administered in combination with everolimus in accordance with the methods of the invention, 85% TGI was achieved. In both models, body weight loss was less than 5% in both the monotherapy and combination therapy treatment groups, consistent with minimal or no drug toxicity.

Thus, in various embodiments of the invention TH-302, or another compound of Formula I, is administered in combination with everolimus to treat cancer. Everolimus is approved for once-a-day administration, and in various embodiments of the combination therapies of the invention, everolimus is administered at an FDA approved dosing amount at a frequency of once per day, and in many of these embodiments, a patient receives multiple doses over a period of at least five day and often for several weeks to several months or longer, of everolimus. As described more fully below, TH-302 and the other compounds of Formula I can be conveniently administered at a frequency of no more than once per week, and in various embodiments of the invention, TH-302 or another compound of Formula I is administered in combination with everolimus administered as described above, and TH-302 is administered no more frequently than once per week. In various of these embodiments, TH-302 or another compound of Formula I is administered in cycles of four weeks, in which TH-302 is administered once per week for three consecutive weeks and is not administered in the fourth week. In various of these embodiments, TH-302 or another compound of Formula I is administered in cycles of three weeks, where TH-302 is administered once per week for two consecutive weeks and is not administered in the third week or where TH-302 is administered only in the first week of the three week cycle. As described below, however, TH-302 can be administered in combination with everolimus more frequently than once per week as well. In any of these embodiments, where the compound is TH-302, the dose can be as described below, i.e., 240-670 mg/m². When everolimus and TH-302 or another compound of Formula I are given on the same day, then TH-302 or other compound of Formula I will typically be given first, and the everolimus will not be administered for at least 30 minutes, and often for at least 2 to at least 4 hours, after administration of the TH-302 or other compound of Formula 1 has stopped. In any of the foregoing embodiments, the cancer can be RCC or neuroblastoma.

As described in Example 3, below, similarly impressive results were obtained by coadministration of the mTOR inhibitor temsirolimus, which has also been approved for use in treating RCC, in combination with TH-302 in accordance with the methods of the invention. In the 786-0 model, temsirolimus monotherapy provided a TGI of 113%, while combination therapy provided a TGI of 137-142% (there was tumor regression). Treatment with temsirolimus alone resulted in 3% body weight loss, and the combination therapy resulted in 8-9% body weight loss. Body weight returned to normal when treatment stopped. In the Caki-1 model, the TGI for combination therapy was 100-101%, compared to TGI for TH-302 or temsirolimus monotherapy under 90%. Similarly to the results in the 786-0 model, the maximal body weight loss in the combination therapy treatment group was 7-9% and returned to normal when the treatment was stopped.

Thus, in various embodiments of the invention TH-302, or another compound of Formula I, is administered in combination with temsirolimus to treat cancer. Temsirolimus is approved for once-a-week administration, and in various embodiments of the combination therapies of the invention, temsirolimus is administered at an FDA approved dosing amount no more frequently than once per week, and in many of these embodiments, a patient receives multiple doses, over a period of many weeks to several months or longer, of temsirolimus. As described more fully below, TH-302 and the other compounds of Formula I can be conveniently administered at a frequency of no more than once per week, and in various embodiments of the invention, TH-302 or another compound of Formula I is administered in combination with temsirolimus administered as described above, and both compounds are administered no more frequently than once per week. In various of these embodiments, TH-302 or another compound of Formula I is administered in cycles of four weeks, in which TH-302 is administered once per week for three consecutive weeks and is not administered in the fourth week. In various of these embodiments, TH-302 or another compound of Formula I is administered in cycles of three weeks, where TH-302 is administered once per week for two consecutive weeks and is not administered in the third week or where TH-302 is administered only in the first week of the three week cycle. In any of these embodiments, where the compound is TH-302, the dose can be as described below, i.e., 240-670 mg/m². When temsirolimus and TH-302 or another compound of Formula I are given on the same day, then TH-302 or other compound of Formula I will typically be given first, and the temsirolimus will not be administered for at least 30 minutes, and often for at least 2 to at least 4 hours, after administration of the TH-302 or other compound of Formula 1 has stopped. In any of the foregoing embodiments, the cancer can be RCC or neuroblastoma.

Example 4 below describes studies in RCC animal models showing that administration of either everolimus or temsirolimus significantly increases tumor hypoxia relative to administration of vehicle control. TH-302 reduces the size of the hypoxic region in the tumor in the Caki-1 model but not in the 786-0 model. Interestingly, co-administration of either mTOR inhibitor in combination with TH-302 reduces tumor hypoxia relative to that caused by either everolimus or temsirolimus alone.

Animal model studies also demonstrate that the combination therapies of the present invention are highly efficacious in the treatment of neuroblastoma. As described in Example 2, below, in the ectopic SK-N-BE(2) neuroblastoma model, TH-302 or everolimus alone demonstrated 45% and 40% TGI, respectively, while the combination therapy achieved 64% TGI. Importantly, body weight loss, a toxicity indicator, was minor (<5%) in all groups tested, indicating no significant added toxicity for the combination therapy relative to the monotherapy.

Collectively, the results of these studies demonstrate that TH-302 and other compounds of Formula I can be administered in combination with an mTOR inhibitor to improve efficacy in any cancer therapy in which mTOR inhibitors provide therapeutic benefit.

### Hypoxia Activated Prodrug Administration

In one aspect, the present invention provides a method of treating cancer comprising administering a therapeutically effective amount of a hypoxia activated prodrug of Formula 1 and a therapeutically effective amount of an mTOR inhibitor to a patient in need of such treatment thereby treating the cancer. In one embodiment, the combination therapy is administered to a patient that has been previously treated with an mTOR inhibitor or a hypoxia activated prodrug of Formula 1, but the cancer is progressing despite the therapy, or the therapy has been discontinued due to cancer progression. In another embodiment, the combination therapy is administered to a patient that has been previously treated with an anti-cancer drug(s) other than an mTOR inhibitor and/or a hypoxia activated prodrug, but the cancer is progressing despite the therapy. In another embodiment, the combination therapy is administered to a cancer patient that has not been previously treated with an anti-cancer drug. As described below, a variety of dosing schedules can be used in accordance with the invention. In various embodiments, when the mTOR inhibitor and TH-302 or another compound of Formula I are given on the same day, then TH-302 or other compound of Formula I will typically be given first, and the mTOR inhibitor will not be administered for at least 30 minutes, and often for at least 2 to at least 4 hours, after administration of the TH-302 or other compound of Formula 1 has stopped.

In various embodiments, the hypoxia activated prodrug of Formula I is selected from the group consisting of TH-281, TH-302, and TH-308. In one embodiment, the hypoxia activated prodrug administered is TH-302. In various embodiments, the TH-302 or other hypoxia activated prodrug of Formula 1 is administered no more frequently than once daily, once every 3 days, once weekly, once every 2 weeks, or once every 3 weeks. In various embodiments, the TH-302 or other hypoxia activated prodrug of Formula 1 is administered parenterally. In various embodiments, the TH-302 or other hypoxia activated prodrug is administered orally (see PCT application no. PCT/US2012/033671, filed Apr. 13, 2011, incorporated herein by reference).

In one embodiment, the hypoxia activated prodrug is TH-302, which is administered in a daily dose of about 240 mg/m² to about 670 mg/m². Suitable administration schedules for doses of TH-302 in this range include the following:

once a week at 575 mg/m²;
once every three weeks at 670 mg/m²;
days one and eight of a twenty-one day cycle at 300, 340, or 480 mg/m²;
days one, eight, and fifteen of a twenty-one day cycle at 240 or 340 mg/m²;
days one, four, eight, and eleven of a twenty-one day cycle at 240 to 480 mg/m²;
days one to five of a twenty-one day cycle at 460 mg/m²;
days one, eight, and fifteen of a twenty-eight day cycle at 240 to 575 mg/m²;
days eight, fifteen, and twenty-two of a twenty-eight day cycle at 240 to 575 mg/m², e.g. 480 mg/m², where mTOR inhibitor administration is initiated on day one; and
once every two weeks at 240 to 670 mg/m², which may follow surgery.
Each of the above schedules can be considered a “cycle” of therapy. Patients will generally receive more than one cycle of therapy, although there may breaks of at least a day, and more generally a week or longer, between each cycle of therapy. Other compound of Formula I are generally dosed in accordance with the above schedules and amounts, with the amount adjusted to reflect how active the compound is relative to TH-302.

When an mTOR inhibitor is combined with a hypoxia activated prodrug according to the present invention, the mTOR inhibitor is administered in amounts and dosing frequencies as disclosed herein, or in amounts and frequencies apparent to the skilled artisan in view of this disclosure, including but not limited to administrations in amounts and frequencies approved for cancer therapy by a regulatory agency such as the FDA.

In various embodiments, the patient's cancer treated is a metastatic cancer or a refractory and/or relapsed cancer, which may have been refractory to first, second, or third line treatment. In various embodiments, the treatment method of the invention is administered as a first, a second, or third line treatment. As used herein, the phrase “first line” or “second line” or “third line” refers to the order of treatments received by a patient. First line treatment are the first treatments given after diagnosis, whereas second or third line treatments are given after the first line treatment or after the second line treatment, respectively. Therefore, first line treatment is the initial treatment for a disease or condition. In patients with cancer, first line, treatment can be surgery, chemotherapy, radiation therapy, or a combination of these therapies. First line treatment is also referred to as primary therapy or primary treatment. Typically, a patient is given a subsequent chemotherapy regimen if the patient does not show a positive clinical response or only shows a marginally positive response to first line treatment, or the first line treatment has stopped after a positive response but the cancer recurs. In this context, “chemotherapy” is used in its broadest sense to incorporate not only classic cytotoxic chemotherapy but also molecularly targeted therapies and immunotherapies.

In another aspect, the treatment methods of the present invention are used for treating hyperproliferative diseases other than cancer.

Methods of preparation of and pharmaceutical compositions of hypoxia activated prodrugs, and other methods of treating cancer by administering various hypoxia activated prodrugs of Formula I are described in Duan et al., J. Med. Chem. 2008, 51, 2412-2420, and PCT Pub. Nos. 2007/002931, 2008/083101, 2010/048330, 2012/006032, and 2012/009288 and PCT Patent Application Nos. PCT/US2012/031677 and PCT/US2012/033671, each of which is incorporated herein by reference.

Other methods of treating cancers, which may be used in combination with the methods of the present invention, are known to one of skilled in the art, and are described, for example, in the product descriptions found in the 2010 or more current edition of the Physician's Desk Reference, Medical Economics Company, Inc., Oradell, N.J.; Goodman and Gilman's The Pharmacological Basis of Therapeutics., Eds. Hardman et al., McGraw-Hill. New York. (US) 2011, 12th Ed., and in publications of the U.S. Food and Drug Administration and the NCCN Guidelines (National Comprehensive Cancer Network). Such methods can be appropriately modified by one of skill in the art, in view of this disclosure, to practice the treatment methods of the present invention.

In one embodiment, the TH-302 is provided in 100 mg vials, lyophilized, and dissolved in D5W and administered intravenously (i.v.) over approximately 30-60 minutes via an infusion pump. The infusion volume depends on the total dose given (in mg) during the infusion. If a dose of less than about 1000 mg is infused, then about 500 mL of D5W are used for infusion. If the dose is greater than about 1000 mg, then about 1000 mL of D5W are used for infusion. In various embodiments in which TH-302 (or another compound of Formula I) and the mTOR inhibitor are administered to a patient on the same day, the TH-302 (or other compound of Formula I) is administered first, and the mTOR inhibitor is administered only after a delay of about 30 minutes to 4 hours or longer.

mTOR Inhibitor Administration

Any mTOR inhibitor can be administered in combination with a hypoxia activated prodrug in accordance with the present invention. In various embodiments, the hypoxia activated prodrug is a compound of Formula I. In various embodiments the compound of Formula I is TH-302. Various illustrative and non-limiting mTOR inhibitors and certain of their illustrative and non-limiting dosing schedules useful in accordance with the present invention is described below.

AZD8055 is a selective and orally bioavailable ATP-competitive mTOR kinase inhibitor with an IC₅₀ of 0.8 nM that is useful in the methods and compositions of the present invention. It has been administered to cancer patients orally at 10, 20, 40, 60, 90, or 120 mg twice daily (BD), and may be administered to patients at such doses and frequency in the methods of the present invention. See, Banerji et al., J. Clin. Oncol. 29: 2011 (suppl; abstr 3096), incorporated herein by reference. For treating brain cancers such as glioblastoma multiforme, anaplastic astrocytoma, anaplastic oligodendroglioma, malignant glioma, and brainstem glioma, AZD8055 can be administered at 120 mg once daily, and this dose is suitable for use in the methods of the invention. Within this treatment regimen, patients may be treated differently depending on whether they are in surgical or nonsurgical treatment groups. Patients in the nonsurgical treatment group can, for example, take AZD8055 by mouth daily for a 42-day cycle of treatment. Patients in the surgical treatment group can take AZD8055 by mouth daily for 7 days, and then have tumor removal surgery; then, at least 3 weeks after surgery, patients resume doses of AZD8055 and continue to take the drug, and a hypoxia-activated prodrug such as TH-302 can be administered in combination in either the pre-surgery treatment period or the post-surgery treatment period or both. Accordingly, in one embodiment of the present invention, AZD8055 is administered in a daily amount of up to 120 mg in combination with a hypoxia activated prodrug of Formula I, including but not limited to TH-302, administered as described herein. In various embodiments of the methods of the invention, the daily amount of AZD8055 is 10, 20, 40 60, 90, or 120 mg. In various embodiments of the methods of the invention, AZD8055 is administered daily.

BEZ235 (NVP-BEZ235) is an inhibitor of PI3K and mTOR (IC₅₀<6 nM) suitable for use in the methods and compositions of the invention. For treating cancer, it has been administered once daily as a gelatin capsule in doses ranging from 10 mg to 1100 mg (see, Burris et al., J. Clin. Oncol. 28:15s, 2010 (suppl; abstr 3005), incorporated herein by reference), and this unit dose form and these dosage amounts can be used in accordance with the methods of the invention. BEZ235 can also be administered as a SDS (solid dispersion system) sachet once daily at 800 mg, 1000 mg, 1400 mg, and 1600 mg in accordance with the methods of the invention. Accordingly, in various embodiments of the present invention, BEZ235 is administered in a daily amount of up to 1600 mg. In various embodiments, the daily amount of BEZ235 is 800, 1000, 1400, or 1600 mg when coadministered with a hypoxia activated prodrug of Formula I, including but not limited to TH-302. In other embodiments, the daily amount of BEZ235 is 100, 200, 300, 400, 500, 600, or 700 mg when administered in combination with a hypoxia activated prodrug of Formula I, including but not limited to TH-302, administered as described herein. In various embodiments of the methods of the invention, AZD8055 is administered daily.

Deforolimus (ridaforolimus) is a small-molecule inhibitor of mTOR suitable for use in the methods and compositions of the invention. Deforolimus can be administered in a dose of 12.5 mg, for example, to treat advanced and pretreated sarcomas, as a 30-minute intravenous infusion once daily for 5 days every 2 weeks. Accordingly, in one embodiment of the present invention, deforolimus is administered in an amount of 12.5 mg in combination with a hypoxia activated prodrug of Formula I, including but not limited to TH-302. In various embodiments, deforolimus is administered once daily in combination with a hypoxia activated prodrug of Formula I, including but not limited to TH-302, administered as described herein. In various embodiments, deforolimus is administered once daily for at least 5 days every 2 weeks.

Everolimus (RAD001), also known as SDZ-RAD, certican, zortress, or afinitorm is an mTOR inhibitor with IC₅₀ of 0.6 nM that is suitable for use in the methods and compositions of the invention. Everolimus can be administered at 5 mg/day, 10 mg/day, or 20 mg/day, typically, 10 mg/day, once daily for treating advanced progressive neuroendocrine tumors of pancreatic origin or renal cell carcinoma in combination with a hypoxia activated prodrug of Formula I, including TH-302. Everolimus can also be administered in accordance with the methods of the invention for treating subependymal giant cell astrocytoma at an initial dose based on body surface area with subsequent titration to attain trough concentrations of 5-10 ng/mL. Everolimus is commercially available as 2.5 mg, 5 mg, 7.5 mg and 10 mg tablets. Such tablets include as excipients butylated hydroxytoluene, magnesium stearate, lactose monohydrate, hypromellose, crospovidone, and anhydrous lactose. Accordingly, in one embodiment of the present invention, everolimus is administered daily, in a daily amount of 5-20 mg, in combination with administration of a compound of Formula I, such as TH-302, administered as described herein. In various embodiments of the methods of the invention, the daily amount of everolimus administered is 5, 10, 15, or 20 mg. In various embodiments of the methods of the invention, everolimus is administered daily.

OSI-027 is another mTOR inhibitor suitable for use in the methods and compositions of the present invention. Patients with advanced solid tumors or lymphoma have received varying doses of OSI-027 in 3 schedules, days 1-3 q7d (51), once weekly (S2), and continuous once daily (S3), with S1 and S2 schedule patients dosed at 10, 15, and 20 mg, and S3 patients dosed at 5, 10, and 20 mg (see Tan et al., J. Clin. Oncol. 28:15s, 2010 (suppl; abstr 3006), incorporated herein by reference). Accordingly, in one embodiment of the present invention, OSI-027 is administered in a daily amount of up to 20 mg in combination with a hypoxia activated prodrug of Formula I, including but not limited to TH-302, administered as described herein. In various embodiments of the methods of the invention, the daily amount of OSI-027 administered is 5, 10, 15, or 20 mg. In various embodiments of the methods of the invention, OSI-027 is administered daily, or administered 3 times a week, or administered once weekly.

Rapamycin (sirolimus), also known as rapamune, is an mTOR inhibitor useful in the methods and compositions of the present invention. Rapamycin can be administered to cancer patients in doses of 10, 20, 30, and 60 mg administered once a week in combination with a hypoxia activated prodrug of Formula I, including but not limited to TH-302, administered as described herein. For treating renal cell carcinoma, sirolimus can be administered, alone or in combination with erlotinib, as a single 6 mg loading dose and then at 2 mg doses daily, in combination with a hypoxia activated prodrug of Formula I, including but not limited to TH-302, administered as described herein. For treating hepatocellular carcinoma, sirolimus can be administered orally at 30 mg once weekly in combination with a hypoxia activated prodrug of Formula I, including but not limited to TH-302, administered as described herein. For treating mixoid chondrosarcoma, such as, metastatic mixoid G2 or G3 (FNCLCC) chondrosarcoma, sirolimus can be administered (1 mg×3/day) on days 1-21, every 28 days in combination with cyclophosphamide 200 mg/day, orally, on days 1-7 and 15-21, in combination with a hypoxia activated prodrug of Formula I, including but not limited to TH-302, administered as described herein. For treating recurrent glioblastoma multiforme, sirolimus can be administered at 2 mg/day and adjusted to achieve serum levels of 4-12 nanograms/ml and can be co-administered in combination with gefitinib on a continuous oral daily dosing in combination with a hypoxia activated prodrug of Formula I, including but not limited to TH-302, administered as described herein.

Accordingly, in one embodiment of the present invention, sirolimus is administered in a low dose, daily, in combination with a hypoxia activated prodrug of Formula I, including but not limited to TH-302, administered as described herein. The daily amount of sirolimus in such a low dose therapy can be, for example and without limitation, 2-3 mg. Thus, the daily amount of sirolimus administered may be 2 or 3 mg. In another embodiment, sirolimus is administered in a high daily dose, once weekly, in combination with a hypoxia activated prodrug of Formula I, including but not limited to TH-302, administered as described herein. The daily amount of sirolimus in such a high dose therapy is in a range of from about 5 mg up to about 60 mg. In various embodiments, the daily amount of the high dose therapy is 10 mg, 20 mg, 30 mg, or 60 mg.

Temsirolimus (torisel) is an mTOR inhibitor suitable for use in the methods and compositions of the invention. Temsirolimus can be administered, for example, to treat renal cell carcinoma, at a dose of 25 mg infused over a 30-60 minute period once a week in combination with a hypoxia activated prodrug of Formula I, including but not limited to TH-302, administered as described herein. Torisel is typically formulated at 25 mg/mL and diluted before administration. Temsirolimus can be administered once weekly in combination with docetaxel administered every 3 weeks in patients with refractory solid malignancies, in combination with a hypoxia activated prodrug of Formula I, including but not limited to TH-302, administered as described herein, where the following amounts of temsirolimus and docetaxel are administered: 60 mg/m² docetaxel/15 mg/m² temsirolimus, 60 mg/m² docetaxel/25 mg/m² temsirolimus, and 50 mg/m² docetaxel/15 mg/m² temsirolimus. For Ewing's sarcoma, temsirolimus can be administered at 25 mg. i.v., weekly, in four week cycles, alone or in combination with cixutumumab 6 mg/kg, administered i.v. once weekly, with restaging after eight weeks, in combination with a hypoxia activated prodrug of Formula I, including but not limited to TH-302, administered as described herein. For treating castration-resistant metastatic prostate cancer, temsirolimus can be administered at 25 mg, i.v., once weekly, and upon PSA/CTC progression, in combination with an anti-androgen, and in either mode, in combination with a hypoxia activated prodrug of Formula I, including but not limited to TH-302, administered as described herein. Accordingly, in one embodiment of the present invention, temsirolimus is administered in a daily amount of 25 mg once weekly in combination with a hypoxia activated prodrug of Formula I, including but not limited to TH-302, administered as described herein. In various embodiments, temsirolimus is administered in a daily amount of 25 mg/m² or about 35 mg once weekly.

XL765 is a mixed mTOR/PI3k inhibitor with IC₅₀ values of 157, 39, 113, 9, and 43 nM for mTOR, p110α, β, γ and δ, respectively, suitable for use in the methods and compositions of the invention. XL765 can be administered to cancer patients once daily (for example at 70, 90, and 100 mg unit doses) and twice daily (for example at 15, 30, 50, and 60 mg unit doses) for 28 day cycles in combination with a hypoxia activated prodrug of Formula I, including but not limited to TH-302, administered as described herein. Accordingly, in one embodiment of the present invention, XL765 is administered in a daily amount of up to 100 mg in combination with a hypoxia activated prodrug of Formula I, including but not limited to TH-302, administered as described herein. In various embodiments, XL765 is administered in a daily amount of 15, 30, 50, 70, or 90 mg. In various embodiments, XL765 is administered once daily. In other embodiments, XL765 is administered twice daily.

The following compounds are other mTOR inhibitors useful in the methods and compositions of the present invention. Chrysophanic acid (chrysophanol) is an EGFR/mTOR pathway inhibitor. GSK1059615 is a pan-PI3K reversible inhibitor, with IC₅₀ values of PI3Kα (0.4 nM), (0.6 nM), γ (5 nM), δ (2 nM), and mTOR (12 nM). GSK2126458 is a PI3K and mTOR inhibitor with a Ki of 19 pM for PI3K. KU-0063794 is an mTOR inhibitor with an IC₅₀ of 10 nM for mTORC 1 and mTORC2, respectively. NU7441 is a selective DNA-dependent protein kinase (DNA-PK) inhibitor with IC₅₀ values of 0.01, 1.7 and 5 μM for DNA-PK, mTOR and PI3-K, respectively. Palomid 529 (P529) is a antitumor PI3K/Akt/mTOR inhibitor with a GI₅₀ of <35 μM in the NCI-60 cell lines panel. PI-103 is a cell-permeable, ATP-competitive PI3K family members inhibitor with IC₅₀ values of 2, 8, 20, 26, 48, 83, 88, 150 nM for DNA-PK, p110α, mTORC1, PI3-KC2β, p110δ, mTORC2, p110β, and p110γ, respectively. PKI-587 is a highly dual PI3K/mTOR kinase inhibitor with IC₅₀ of 0.4 nM and <0.1 μM for PI3K-α and mTOR, respectively. PP242 is a selective mTOR inhibitor with an IC₅₀ of 8 nM. WAY-600 is a ATP-competitive mTOR inhibitor with an IC₅₀ of 9 nM. WYE-125132 is an ATP-competitive and specific mTOR kinase inhibitor with an IC₅₀ of 0.19 nM. WYE-354 is a mTOR inhibitor with an IC₅₀ of 5 nM. WYE-687 is an ATP-competitive mTOR inhibitor with an IC₅₀ of 7 nM.

Thus, in accordance with the present invention, TH-302 or another compound of Formula I is co-administered with an mTOR inhibitor, optionally in combination with other treatments. A synergistic effect may be achieved by using more than one compound in a pharmaceutical composition of the invention, i.e. a compound of Formula I is combined with at least another agent as active ingredient, which is either another compound of Formula I, or an mTOR inhibitor, or both, or another anti-cancer agent. The active ingredients useful in the methods of the invention can be used either simultaneously (as in an admixed formulation) or sequentially. Thus, the invention also relates to a compound or pharmaceutical composition for inhibiting abnormal cell growth or cancer in a mammal which comprises an amount of a compound of Formula I, or a pharmaceutically acceptable salt or solvate or prodrug thereof, in combination with an amount of another mTOR inhibitor (and optionally another anti-cancer therapeutic), wherein the amounts of the compound, salt, solvate, or prodrug, and of the mTOR inhibitor (and of the another anti-cancer therapeutic) are together effective in inhibiting abnormal cell growth or cancer in a patient. The combination therapies described herein are thus suitable for use in combination with known anti-cancer agents.

The invention also relates to a set of items, which may be packaged into a kit, consisting of separate packs of an effective amount of a compound of Formula I and an mTOR inhibitor (or pharmaceutically acceptable salts, derivatives, solvates, and stereoisomers thereof, including mixtures thereof in all ratios, and optionally an effective amount of a further medicament active ingredient). The set or kit comprises suitable containers, such as boxes, individual bottles, bags, or ampoules. The set may, for example, comprise separate ampoules, each containing an effective amount of a compound of Formula I and an mTOR inhibitor (or pharmaceutically acceptable salts, derivatives, solvates, and stereoisomers thereof, including mixtures thereof in all ratios, and optionally an effective amount of a further medicament active ingredient), each in dissolved or lyophilized form. The set or kit of the invention may also contain an article that contains written instructions or directs the user to written instructions that explain the how the compounds are administered in accordance with the invention to treat a disease, such as cancer.

The invention also relates to the use of compounds of Formula I and mTOR inhibitor compounds and/or physiologically acceptable salts thereof for the prophylactic or therapeutic treatment and/or monitoring of diseases, such as cancer, that are caused, mediated, and/or propagated by abnormal cellular proliferative activity.

Furthermore, the invention relates to the use of compounds of Formula I and mTOR inhibitors and/or physiologically acceptable salts thereof for the production of a medicament for the prophylactic or therapeutic treatment and/or monitoring of diseases, such as cancer, that are caused, mediated, and/or propagated by abnormal cellular proliferative activity.

Compounds of Formula I and mTOR inhibitors and/or a physiologically acceptable salt thereof can also be employed as intermediates for the preparation of further medicament active ingredients. The medicament is preferably prepared in a non-chemical manner, e.g. by combining the active ingredient with at least one solid, fluid and/or semi-fluid carrier or excipient, and optionally in conjunction with a single or more other active substances in an appropriate dosage form.

Another object of the present invention is compounds of Formula I and mTOR inhibitors according to the invention and/or physiologically acceptable salts thereof for use in the prophylactic or therapeutic treatment and/or monitoring of diseases, such as cacner, that are caused, mediated, and/or propagated by abnormal cellular proliferative activity. Another preferred object of the invention concerns compounds of Formula I and mTOR inhibitors according to the invention and/or physiologically acceptable salts thereof for use in the prophylactic or therapeutic treatment and/or monitoring of hyperproliferative disorders, including cancer.

The prior teaching of the present specification concerning the compounds of Formula I and mTOR inhibitors, including any preferred embodiment thereof, is valid and applicable without restrictions to the compounds Formula I and mTOR inhibitors and their salts for use in the prophylactic or therapeutic treatment and/or monitoring of hyperproliferative disorders.

As will be apparent to the skilled artisan upon reading this disclosure, the mTOR inhibitors employed in the methods and compositions of the invention can be formulated in various ways known to the skilled artisan. Exemplary formulations of certain mTOR inhibitors are known in the art and are commercially available.

### Examples

### Example 1

### In Vivo Activity of TH-302 in Combination with Everolimus for Treating RCC

Two renal cell carcinoma (RCC) ectopic xenograft models were established by subcutaneous implantation of Caki-1 or 786-0 cells into the flanks of nude mice. When tumor size was approximately 150 mm³, animals were treated with everolimus (5 mg/kg, QD×19, p.o.), TH-302 (50 mg/kg, QD×5/week×3 weeks, i.p.), or both everolimus and TH-302. In the combination groups, both drugs were administered on day 1. In the Caki-1 model, 87% TGI was observed in combination group versus 52% TGI from everolimus monotherapy or 57% TGI from TH-302 monotherapy. A pharmacodynamic study using immunohistochemistry showed that after 7 days treatment of everolimus, cell proliferation (as measured by the expression of the nuclear antigen Ki67), microvessel density (by the angiogenic marker CD31) and phosphorylation of the S6 ribosomal protein (p-S6) were significantly decreased, consistent with increased hypoxia in the tumor tissue. In the 786-0 model, everolimus monotherapy gave 49% TGI, while TH-302 monotherapy did not demonstrate statistically significant efficacy. However, when TH-302 was combined with everolimus, a TGI of 85% was achieved. In both models, body weight loss was less than 5% in both the monotherapy and combination therapy treatment groups, consistent with minimal or no drug toxicity. In contrast to the in vivo additivity of combination therapy seen in these in vivo models, TH-302 exhibits no additive effect with everolimus in in vitro cytotoxicity assays. Cells were treated with TH-302 and 10-20 μM of everolimus for 2 hours under under either normoxia or hypoxia. After a wash to remove drug, cells were incubated with fresh media at 37° C. for an additional 3 days in the presence of everolimus. Cell viability was determined using AlamarBlue. This observation is consistent with additivity in the microenvironment, rather than cancer cell autonomous synergy.

### Example 2

### In Vivo Activity of TH-302 in Combination with Everolimus for Treating Neuroblastoma

Antitumor activity of TH-302 in combination with everolimus was demonstrated in ectopic neuroblastoma produced by implantation of SK-N-BE(2) cells. When tumor size was approximately 150 mm³, animals were treated with everolimus (5 mg/kg, QD×19, oral), TH-302 (50 mg/kg, QD×5/week×3 weeks, i.p.), or both everolimus and TH-302. The administration of everolimus and TH-302 was started on the same day. TH-302 or everolimus monotherapy demonstrated 45% and 40% TGI, respectively, while the combination therapy achieved 64% TGI. Importantly, body weight loss, a toxicity indicator, was very minor (<5%) in all groups tested and was not significantly increased with TH-302 in combination with everolimus. TH-302 exhibits no additive effect with everolimus in in vitro cytotoxicity assays.

### Example 3

### In Vivo Activity of TH-302 in Combination with Temsirolimus for Treating RCC

Renal cell carcinoma (RCC) ectopic xenografts were established by subcutaneous implantation of Caki-1 or 786-0 cells into the flanks of nude mice. When tumor size was approximately 150 mm³, animals were treated with temsirolimus (20 mg/kg, QD×19, i.p.), TH-302 (50 mg/kg, QD×5/week×2 to 3 weeks, i.p.), or both temsirolimus and TH-302 in two different schedules. In one combination therapy group, the temsirolimus and TH-302 administration both began on day 1; in the other combination therapy group, the TH-302 administration was initiated on day 8 after the first temsirolimus administration. Thus, two TH-302 monotherapy groups served as comparison groups (one group starting on day 1 and the other starting on day 8). In the 786-0 model, temsirolimus showed significant inhibition as monotherapy, providing TGI at 113%; surprisingly, the antitumor activity was increased in the combination therapy group to a TGI 137-142%. In the Caki-1 model, 102% TGI was observed in the day 1 combination therapy group, and 101% TGI was observed in the day 8 combination therapy group, which compares favorably with the 79% TGI from the temsirolimus monotherapy group, 79% TGI from the TH-302 day 1 monotherapy group, and 89% from the TH-302 day 8 monotherapy group. In both models, temsirolimus monotherapy resulted in less than 5% maximal body weight loss, and combination therapy with TH-302 resulted in 8-10% body weight loss. The weight returned to normal when treatment stopped.

### Example 4

### TH-302 Reduces mTOR Inhibitor Induced Hypoxia in RCC Xenograft Tumors

Nude mice bearing 786-0 or Caki-1 RCC xenograft tumors were randomized into 4 groups (6-8 mice/group): vehicle, mTOR inhibitor, TH-302 monotherapy, and TH-302 in combination with mTOR inhibitor, respectively. In the mTOR inhibitor monotherapy group, animals were dosed with a QD×8 regimen, and tumors were sampled 4 hr after the last treatment. In the TH-302 monotherapy group, 150 mg/kg was administered intraperitoneally to the animals, and the tumors were sampled 72 hr after the TH-302 treatment. In the combination therapy group, TH-302 was administered 4 hr after the last dose of mTOR inhibitor (QD×8, oral), and the tumors were sampled 72 hr later. Pimonidazole was administered 1 hr before euthanization (tumors were sampled on euthanization).

A dose of 5 mg/kg of everolimus was orally administered in the 786-0 xenograft model. Based on microscopic evaluation of the harvested tissues, everolimus increased hypoxia in the tumor. By morphometric analysis, the tumor hypoxic fraction was 6.3±1.5% in the everolimus monotherapy group, as compared to 2.3±0.8% in the vehicle treated group. The tumor hypoxic fraction in the TH-302 monotherapy group was 3.7±1.2%. When TH-302 was administered in combination with everolimus, the tumor hypoxic fraction was 5.0±1.7%.

A dose of 20 mg/kg of temsirolimus was administered intraperitoneally in the Caki-1 xenograft model. A significant increase of tumor hypoxia was observed in the temsirolimus monotherapy group. After temsirolimus treatment, the tumor hypoxic fraction was 12.8±0.7%, as compared to 7.5±1.9% in vehicle treated group (p<0.05). The tumor hypoxic fraction in the TH-302 monotherapy group was 4.3±0.7%. When TH-302 was administered in combination with temsirolimus, the hypoxic fraction was 7.9±0.5%, significantly different from that observed in the temsirolimus monotherapy group (p<0.05).

Although the present invention has been specifically disclosed with respect to certain aspects, embodiments, and optional features, one of skill in the art will appreciate that modification, improvement, and variation of such aspects, embodiments, and optional features can be practiced within the scope of this invention.

1. A method of treating cancer, said method comprising administering to a patient in need of such treatment a therapeutically effective amount of a hypoxia activated prodrug in combination with a therapeutically effective amount of an mTOR inhibitor.

2. The method of claim 1, wherein the hypoxia activated prodrug is a compound of Formula I: 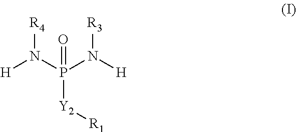
wherein
Y₂ is O, S, NR₆, NCOR₆, or NSO₂R₆;
R₆ is C₁-C₆ alkyl, C₁-C₆ heteroalkyl, aryl, or heteroaryl;
R₃ and R₄ are independently selected from the group consisting of 2-haloalkyl, 2-alkylsulfonyloxyalkyl, 2-heteroalkylsulfonyloxyalkyl, 2-arylsulfonyloxyalkyl, and 2-heteroalkylsulfonyloxyalkyl;
R₁ has the formula L-Z₃;
L is C(Z₁)₂;
each Z₁ independently is hydrogen, halogen, C₁-C₆ alkyl, C₁-C₆ heteroalkyl, aryl, heteroaryl, C₃-C₈ cycloalkyl, heterocyclyl, C₁-C₆ acyl, C₁-C₆ heteroacyl, aroyl, or heteroaroyl;
or L is: 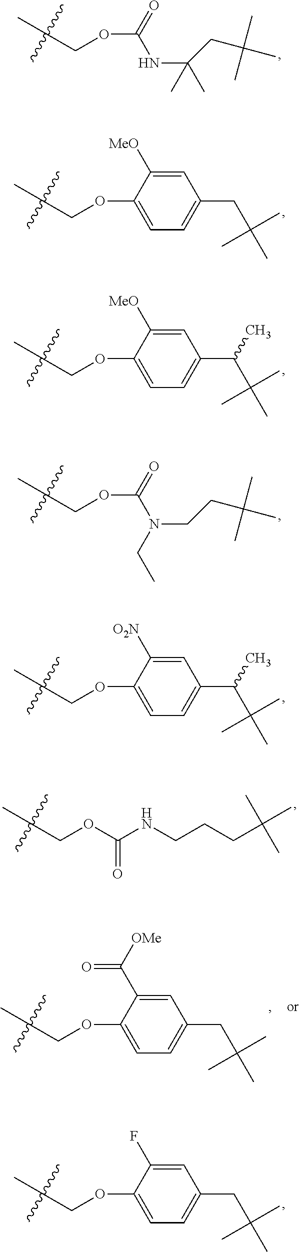
Z₃ is a bioreductive group having a formula selected from the group consisting of: 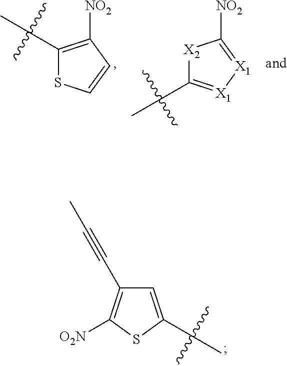
each X₁ is independently N or CR₈;
X₂ is NR₇, S, or O;
each R₇ is independently C₁-C₆ alkyl, C₁-C₆ heteroalkyl, C₃-C₈ cycloalkyl, heterocyclyl, aryl or heteroaryl;
and R₈ is independently hydrogen, halogen, cyano, CHF₂, CF₃, CO₂H, amino, C₁-C₆ alkyl, C₁-C₆ heteroalkyl, C₁-C₆ cycloalkyl, C₁-C₆ alkoxy, C₁-C₆ alkylamino, C₁-C₆ dialkylamino, aryl, CON(R₇)₂, C₁-C₆ acyl, C₁-C₆ heteroacyl, aroyl or heteroaroyl;
or a pharmaceutically acceptable salt thereof.

3. The method of claim 2, wherein said mTOR inhibitor is selected from the group consisting of AZD8055, BEZ235, deforolimus, everolimus, OSI-027, sirolimus, temsirolimus, and XL765.

4. The method claim 3, wherein said hypoxia activated prodrug is TH-302 and said mTOR inhibitor is everolimus or temsirolimus.

5. The method of claim 1, wherein said cancer is selected from the group consisting of brain cancers, castration-resistant metastatic prostate cancer, Ewing's sarcoma, neuroblastoma, neuroendocrine tumors of pancreatic origin, renal cell carcinoma, sarcoma, and subependymal giant cell astrocytoma.

6. The method of claim 4, wherein said cancer is selected from the group consisting of neuroblastoma, neuroendocrine tumors of pancreatic origin, renal cell carcinoma, and subependymal giant cell astrocytoma.

7. A pharmaceutical formulation comprising TH-302, an mTOR inhibitor, and at least one pharmaceutically acceptable excipient.

8. The pharmaceutical composition of claim 7, wherein said mTOR inhibitor is selected from the group consisting of AZD8055, BEZ235, deforolimus, everolimus, OSI-027, sirolimus, temsirolimus, and XL765.

9. An in vivo method of inhibiting growth of a tumor, comprising contacting the tumor with an effective amount of a compound of Formula 1: 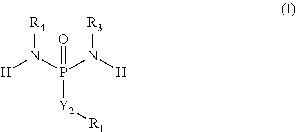
wherein
Y₂ is O, S, NR₆, NCOR₆, or NSO₂R₆;
R₆ is C₁-C₆ alkyl, C₁-C₆ heteroalkyl, aryl, or heteroaryl;
R₃ and R₄ are independently selected from the group consisting of 2-haloalkyl, 2-alkylsulfonyloxyalkyl, 2-heteroalkylsulfonyloxyalkyl, 2-arylsulfonyloxyalkyl, and 2-heteroalkylsulfonyloxyalkyl;
R₁ has the formula L-Z₃;
L is C(Z₁)₂;
each Z₁ independently is hydrogen, halogen, C₁-C₆ alkyl, C₁-C₆ heteroalkyl, aryl, heteroaryl, C₃-C₈ cycloalkyl, heterocyclyl, C₁-C₆ acyl, C₁-C₆ heteroacyl, aroyl, or heteroaroyl;
or L is: 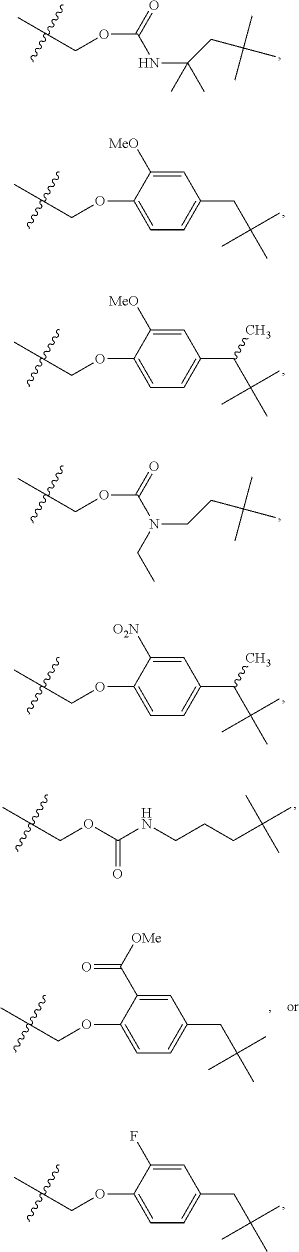
Z₃ is a bioreductive group having a formula selected from the group consisting of: 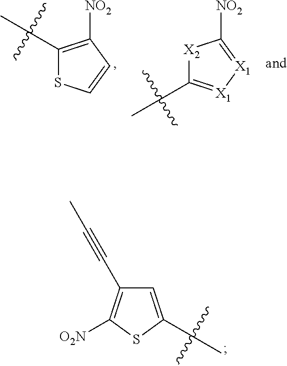
each X₁ is independently N or CR₈;
X₂ is NR₇, S, or O;
each R₇ is independently C₁-C₆ alkyl, C₁-C₆ heteroalkyl, C₃-C₈ cycloalkyl, heterocyclyl, aryl or heteroaryl;
and R₈ is independently hydrogen, halogen, cyano, CHF₂, CF₃, CO₂H, amino, C₁-C₆ alkyl, C₁-C₆ heteroalkyl, C₁-C₆ cycloalkyl, C₁-C₆ alkoxy, C₁-C₆ alkylamino, C₁-C₆ dialkylamino, aryl, CON(R₇)₂, C₁-C₆ acyl, C₁-C₆ heteroacyl, aroyl or heteroaroyl;
or a pharmaceutically acceptable salt thereof
in combination with an mTOR inhibitor.

10. An in vivo method of reducing tumor hypoxia in a tumor treated with an mTOR inhibitor, the method comprising coadministering an effective amount of a compound of Formula (I): 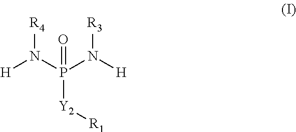
wherein
Y₂ is O, S, NR₆, NCOR₆, or NSO₇R₆;
R₆ is C₁-C₆ alkyl, C₁-C₆ heteroalkyl, aryl, or heteroaryl;
R₃ and R₄ are independently selected from the group consisting of 2-haloalkyl, 2-alkylsulfonyloxyalkyl, 2-heteroalkylsulfonyloxyalkyl, 2-arylsulfonyloxyalkyl, and 2-heteroalkylsulfonyloxyalkyl;
R₁ has the formula L-Z₃;
L is C(Z₁)₂;
each Z₁ independently is hydrogen, halogen, C₁-C₆ alkyl, C₁-C₆ heteroalkyl, aryl, heteroaryl, C₃-C₈ cycloalkyl, heterocyclyl, C₁-C₆ acyl, C₁-C₆ heteroacyl, aroyl, or heteroaroyl;
or L is: 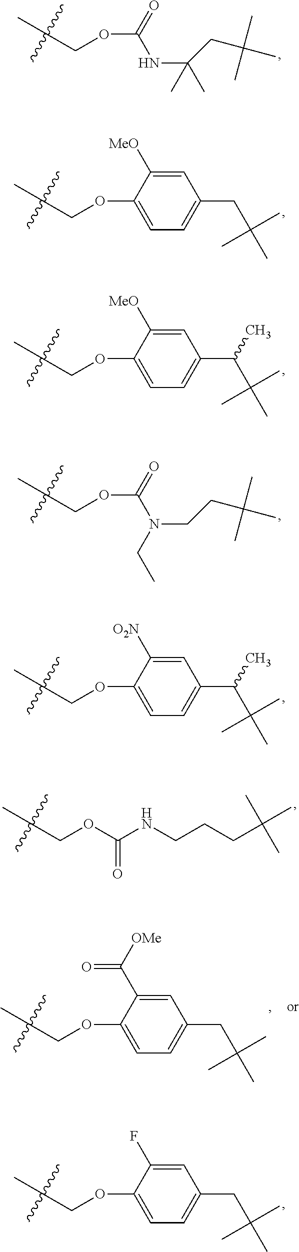
Z₃ is a bioreductive group having a formula selected from the group consisting of: 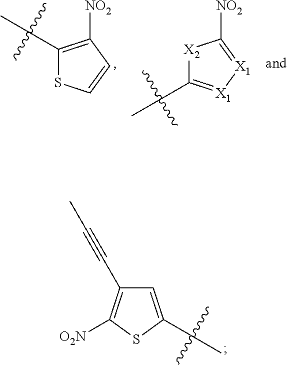
each X₁ is independently N or CR₈;
X₂ is NR₇, S, or O;
each R₇ is independently C₁-C₆ alkyl, C₁-C₆ heteroalkyl, C₃-C₈ cycloalkyl, heterocyclyl, aryl or heteroaryl;
and R₈ is independently hydrogen, halogen, cyano, CHF₂, CF₃, CO₂H, amino, C₁-C₆ alkyl, C₁-C₆ heteroalkyl, C₁-C₆ cycloalkyl, C₁-C₆ alkoxy, C₁-C₆ alkylamino, C₁-C₆ dialkylamino, aryl, CON(R₇)₂, C₁-C₆ acyl, C₁-C₆ heteroacyl, aroyl or heteroaroyl;
or a pharmaceutically acceptable salt thereof
to the tumor.

11. The method of claim 9, wherein the mTOR inhibitor is everolimus or temsirolimus.

12. The method of claim 9, wherein the compound of Formula 1 is TH-302.

13. The method of claim 1, wherein said mTOR inhibitor is selected from the group consisting of AZD8055, BEZ235, deforolimus, everolimus, OSI-027, sirolimus, temsirolimus, and XL765.

14. The method of claim 2, wherein said cancer is selected from the group consisting of brain cancers, castration-resistant metastatic prostate cancer, Ewing's sarcoma, neuroblastoma, neuroendocrine tumors of pancreatic origin, renal cell carcinoma, sarcoma, and subependymal giant cell astrocytoma.

15. The method of claim 3, wherein said cancer is selected from the group consisting of brain cancers, castration-resistant metastatic prostate cancer, Ewing's sarcoma, neuroblastoma, neuroendocrine tumors of pancreatic origin, renal cell carcinoma, sarcoma, and subependymal giant cell astrocytoma.

16. The method of claim 10, wherein the mTOR inhibitor is everolimus or temsirolimus.

17. The method of claim 11, wherein the compound of Formula (I) is TH-302.

18. The method of claim 11, wherein the compound of Formula (I) is TH-302.

19. The method of claim 16, wherein the compound of Formula (I) is TH-302.
